(19) United States
(12) Patent Application Publication
(10) Pub. No.: US 2015/0153295 A1
(43) Pub. Date: Jun. 4, 2015

(54) SENSOR FOR DETECTING PROPERTY OF LIQUID

(71) Applicant: AISAN KOGYO KABUSHIKI KAISHA, Aichi-ken (JP)

(72) Inventor: Masaki Ikeya, Aichi-ken (JP)

(73) Assignee: AISAN KOGYO KABUSHIKI KAISHA, Aichi-ken (JP)

(21) Appl. No.: 14/558,504

(22) Filed: Dec. 2, 2014

(30) Foreign Application Priority Data
Dec. 3, 2013 (JP) 2013-249850

(51) Int. Cl. G01N 27/22; G01N 33/22; G01R 27/26

(52) U.S. Cl. CPC G01N 27/22; G01R 27/2605; G01N 33/22

(57) ABSTRACT

A liquid sensor may comprise a outer member, an inner member, a storage space having a tubular shape defined by the outer member and the inner member, an inflow path disposed on a first side in an axis direction of the storage space, and an outflow path disposed on a second side in the axis direction of the storage space. The inflow path may comprise a center line not passing through a center axis of the storage space in a plane vertical to the axis direction of the storage space such that the liquid inside the storage space forms a spiral flow including a flow from the first side in the axis direction of the storage space to the second side in the axis direction of the storage space and a flow revolving along a circumferential direction of the storage space.

### CROSS-REFERENCE TO RELATED APPLICATION

This application claims priority to Japanese Patent Application No. 2013-249850 filed on Dec. 3, 2013, the contents of which are hereby incorporated by reference into the present application.

### TECHNICAL FIELD

A technology disclosed herein relates to a sensor configured to detect a property (i.e. a characteristic and/or a state) of liquid and, in particular, to a sensor including a tubular storage space configured to store liquid.

### DESCRIPTION OF RELATED ART

Japanese Translation of PCT International Application, No. 2009-505074 A discloses a fluid quality sensor device configured to detect a property of fluid, such as a urea concentration fluid sensor and an alcohol concentration sensor. The sensor device includes a first electrode in which a fluid path is formed, and a second electrode supported in the fluid path. The first electrode and the second electrode function as a cathode and an anode of a capacitor, respectively. The sensor device uses the capacitor to measure a quality of fluid serving as a dielectric material.

### SUMMARY

In the technology, the fluid flows through the fluid path in a linear fashion. In this case, the fluid may be retained in the fluid path, there is a possibility that a property of the fluid may not be properly detected.

This specification provides a technology that makes it possible to properly detect a property of liquid.

The present specification discloses a liquid sensor configured to detect a property of liquid, the liquid sensor may comprise a first outer member, an inner member, a storage space, an inflow path and an outflow path. The first outer member may have a tubular shape. The inner member may be disposed inside the first outer member. The storage space may be configured to store the liquid and having a tubular shape defined by an inner surface of the first outer member and an outer surface of the inner member. The inflow path may be a path through which the liquid flows in from outside the storage space to inside the storage space and disposed on a first side in an axis direction of the storage space. The outflow path may be a path through which the liquid flows out from inside the storage space to outside the storage space and disposed on a second side in the axis direction of the storage space. The inflow path may comprise a center line not passing through a center axis of the storage space in a plane vertical to the axis direction of the storage space such that the liquid inside the storage space forms a spiral flow including a flow from the first side in the axis direction of the storage space to the second side in the axis direction of the storage space and a flow revolving along a circumferential direction of the storage space.

In the sensor, the liquid inside the storage space forms a spiral flow. This configuration makes it possible to better inhibit the liquid from being retained inside the storage space than does the configuration in which the liquid inside the storage space forms a linear flow. Therefore, by using of the sensor, the property of the liquid may be properly detected.

### BRIEF DESCRIPTION OF THE DRAWINGS

FIG. 1 shows schematic configuration of a fuel supplying unit.

FIG. 2 shows a longitudinal cross-sectional view of a liquid property sensor of a first embodiment.

FIG. 3 shows a cross section of FIG. 2.

FIG. 4 shows a IV-IV cross section of FIG. 3.

FIG. 5 shows a longitudinal cross-sectional view of a liquid property sensor of a second embodiment.

FIG. 6 shows a VI-VI cross section of FIG. 5.

FIG. 7 shows a longitudinal cross-sectional view of a liquid property sensor of a third embodiment.

FIG. 8 shows a VIII-VIII cross section of FIG. 7.

FIG. 9 shows a IX-IX cross section of FIG. 7.

FIG. 10 shows a longitudinal cross-sectional view of a liquid property sensor of a fourth embodiment.

FIG. 11 shows a XI-XI cross section of FIG. 10.

FIG. 12 shows a longitudinal cross-sectional view of a liquid property sensor of a fifth embodiment.

FIG. 13 shows a XIII-XIII cross section of FIG. 12.

### DETAILED DESCRIPTION

(Feature 1)

In the plane vertical to the axis direction of the storage space, the inflow path may allow the liquid to flow in from outside the first outer member to inside the storage space. In the plane vertical to the axis direction of the storage space, the center line of the inflow path may intersect the inner surface of the first outer member without intersecting the outer surface of the inner member. In this configuration, the liquid inside the storage space may properly flow in a spiral manner. The liquid may be properly inhibited from being retained inside the storage space.

(Feature 2)

In the plane vertical to the axis direction of the storage space, the outflow path may comprise a center line not passing through the center axis of the storage space. In this configuration, the liquid forming a spiral flow may properly flow out of the storage space. The liquid may be properly inhibited from being retained inside the storage space.

(Feature 3)

The first outer member may comprise a first electrode portion functioning as an electrode. The inner member may comprise a second electrode portion functioning as an electrode. The second electrode portion may oppose the first electrode portion.

(Feature 4)

The liquid sensor may comprise a housing member. The housing member may comprise a housing space disposed on an upper stream side of the storage space and be configured to house the liquid, and an opening through which the liquid flows into the housing space. The opening may be located inside the inner member in the plane vertical to the axis direction of the storage space. The inflow path may have a shape piercing through the housing member. The storage space may communicate with the housing space through the inflow path.

(Feature 5)

The inflow path may have a shape piercing through the first outer member.

(Feature 6)

The outflow path may have a shape piercing through the first outer member.

(Feature 7)

The liquid sensor may comprise a second outer member disposed outside the first outer member and having a tubular shape, and an introducing space defined by an outer surface of the first outer member and an inner surface of the second outer member and having a tubular shape. The storage space may communicate with the introducing space through the inflow path and the outflow path. In this configuration, the second outer member is provided. This makes it possible to inhibit a disturbance to be applied from outside the sensor to the first outer member.

(Feature 8)

The liquid sensor may comprise another one or more inflow paths through which the liquid flows in from outside the storage space to inside the storage space and disposed on the first side in the axis direction of the storage space. All of the inflow paths may be disposed at different positions from one another in the plane vertical to the axis direction of the storage space. This configuration better allows the liquid inside the storage space to properly flow in a spiral manner than does a configuration in which only a single inflow path is provided. This makes it possible to properly inhibit the liquid from being retained inside the storage space.

(Feature 9)

The liquid sensor may comprise another one or more outflow paths through which the liquid flows out from inside the storage space to outside the storage space and disposed on the second side in the axis direction of the storage space. All of the outflow paths may be disposed at different positions from one another in the plane vertical to the axis direction of the storage space. This configuration better allows the liquid forming a spiral flow to properly flow out of the storage space than does a configuration in which only a single outflow path is provided. This makes it possible to properly inhibit the liquid from being retained inside the storage space.

Representative, non-limiting examples of the present invention will now be described in further detail with reference to the attached drawings. This detailed description is merely intended to teach a person of skill in the art further details for practicing preferred aspects of the present teachings and is not intended to limit the scope of the invention. Furthermore, each of the additional features and teachings disclosed below may be utilized separately or in conjunction with other features and teachings to provide improved sensor, as well as methods for using and manufacturing the same.

Moreover, combinations of features and steps disclosed in the following detailed description may not be necessary to practice the invention in the broadest sense, and are instead taught merely to particularly describe representative examples of the invention. Furthermore, various features of the above-described and below-described representative examples, as well as the various independent and dependent claims, may be combined in ways that are not specifically and explicitly enumerated in order to provide additional useful embodiments of the present teachings.

All features disclosed in the description and/or the claims are intended to be disclosed separately and independently from each other for the purpose of original written disclosure, as well as for the purpose of restricting the claimed subject matter, independent of the compositions of the features in the embodiments and/or the claims. In addition, all value ranges or indications of groups of entities are intended to disclose every possible intermediate value or intermediate entity for the purpose of original written disclosure, as well as for the purpose of restricting the claimed subject matter.

### First Embodiment

A fuel feeding unit 1 of the present embodiment is mounted in an automobile, and feeds fuel to an engine (not illustrated). The fuel supplying unit 1 includes a fuel tank 10, a fuel pump unit 30, and a sensor device 2.

The fuel tank 10 retains a fuel. The fuel may be gasoline or mixed fuel of gasoline and ethanol.

The fuel pump unit 30 includes a reserve cup 20, a low-pressure filter 32, a pump body 34, a high-pressure filter 36, a pressure regulator 40, and a discharge port 12. The reserve cup 20, low-pressure filter 32, the pump body 34, the high-pressure filter 36, and the pressure regulator 40 are disposed inside of the fuel tank 10. The discharge port 12 is disposed outside of the fuel tank 10.

The reserve cup 20 is fixed to a set plate 14 of the fuel tank 10 by a prop 22. A jet pump (not illustrated) sends the fuel outside of the reserve cup 20 into the reserve cup 20. The reserve cup 20 stores the fuel sent from the jet pump. The reserve cup 20 stores the low-pressure filter 32, the pump body 34, and the high-pressure filter 36.

The low-pressure filter 32 is formed by a nonwoven fabric and has a bag shape. The low-pressure filter 32 communicates with the suction opening 34a of the pump body 34.

The pump body 34 sucks the fuel stored in the fuel tank 10 through a suction opening 34a of the pump body 34 and pressurizes the fuel Then, the pump body 34 forces the pressurized fuel into the high-pressure filter 36 through an outlet 34b of the pump body 34.

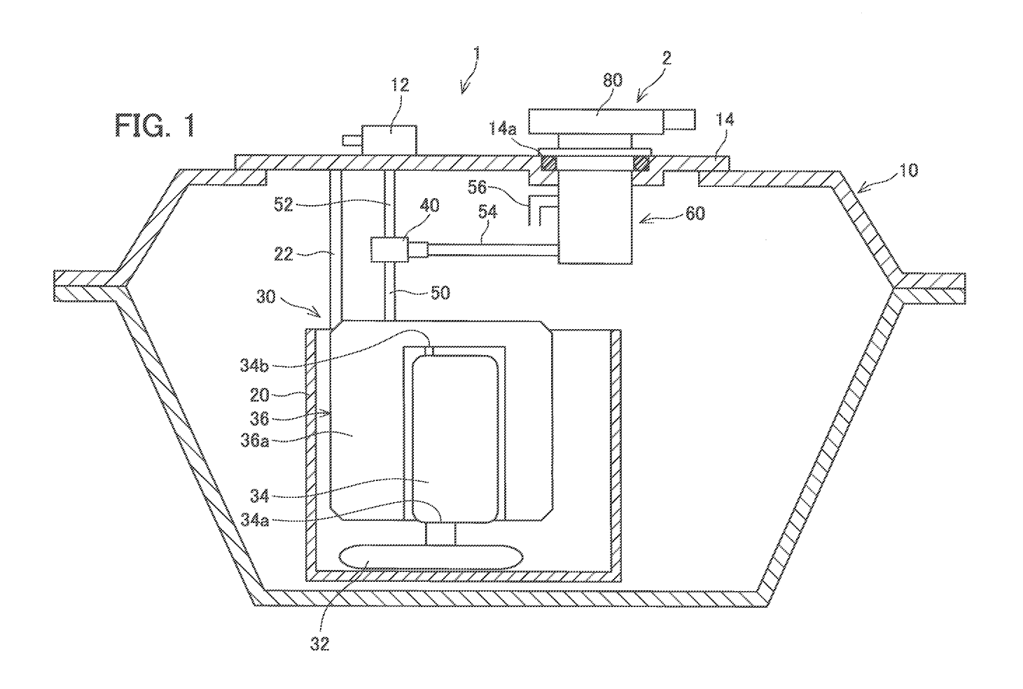

The high-pressure filter 36 includes the case 36a and a filter member (not illustrated). Although illustrated in a simplified manner in FIG. 1, the ease 36a is disposed in such a manner as to extend circumferentially around the pump body 34. The fuel having flowed into the case 36a is filtered by the filter member of and is sent out into the pressure regulator 40 through a pipe 50.

When a pressure of the fuel inside the pressure regulator 40 becomes equal to or higher than a predetermined pressure, the pressure regulator 40 sends extra fuel inside the pipe 50 to a liquid property sensor 60 of the sensor device 2 through a pipe 54. This causes a pressure of the fuel inside the pipe 50 to be adjusted to be a constant pressure. Further, the pressure regulator 40 sends the pressure-adjusted fuel to the discharge port 12 through a pipe 52. As a result of this, the fuel is sent from the discharge port 12 to the engine (not illustrated).

The sensor device 2 is configured to detect a concentration of ethanol contained in fuel. The sensor device 2 includes the liquid property sensor 60 and a control device 80. The liquid property sensor 60 is disposed at an upper end of the fuel tank 10, and is fitted in an opening 14a in the set plate 14.

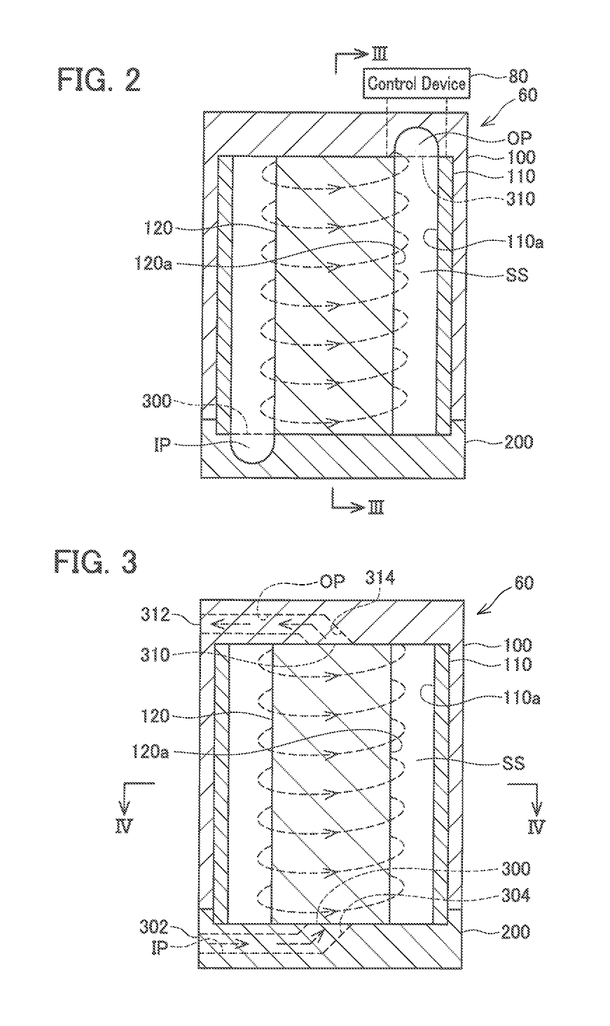

As shown in FIG. 2, the liquid property sensor 60 includes a first casing 100, a second casing 200, an outer electrode 110, and an inner electrode 120. The first casing 100 and the second casing 200 are each formed by a non-conducting material such as resin. The outer electrode 110 and the inner electrode 120 are each formed by a conducting material.

The first casing 100 is disposed on an upper side in a vertical direction (i.e. a direction from top to bottom of FIG. 2). The first casing 100 has a cylindrical shape whose upper end is closed and whose lower end is open. A center axis of the first casing 100 extends in the vertical direction.

The second casing 200 is disposed on a lower side in the vertical direction. The second casing 200 has a substantially disc shape. A center axis of the second casing 200 extends in the vertical direction, and corresponds to the center axis of the first casing 100. Further, a diameter of the second casing 200 is as large as a diameter of the first casing 100. Therefore, connecting the lower end of the first casing 100 to an upper end of the second casing 200 forms a whole casing having a hollow shape.

The second casing 200 includes an inflow path IP through which the fluid flows from outside the liquid property sensor 60 to inside a storage space SS inside the liquid property sensor 60. An end of the inflow path IP is an opening 300 formed in an upper surface of the second casing 200, and the other end of the inflow path IP is an opening 302 (sec FIG. 3) formed in an outer surface of the second casing 200. In a cross-section shown in FIG. 2, the inflow path IP has a circular shape protruding downward. The inflow path IP communicates with the pipe 54 and with the storage space SS. Therefore, the inflow path IP allows fuel supplied from the pipe 54 to flow into the storage space SS.

The first casing 100 includes an outflow path OP through which the liquid flows from the storage space SS inside the liquid property sensor 60 to outside the liquid property sensor 60. An end of the outflow path OP is an opening 310 formed in a lower surface of the first casing 100, and the other end of the outflow path OP is an opening 312 (see FIG. 3) formed in an outer surface of the first casing 100. In the cross-section shown in FIG. 2, the outflow path OP has a circular shape protruding upward. The outflow path OP communicates with an outflow pipe 56 shown in FIG. 1, and communicates with the storage space SS and with an internal space in the fuel tank 100 through the outflow pipe 56. Therefore, the outflow path OP allows the fuel to flow out of the storage space SS into the internal space in the fuel tank 10. It should he noted that the outflow pipe 56 is provided at such a position as to allow the fuel to flow out of the liquid property sensor 60 into the reserve cup 20.

The outer electrode 110 has a cylindrical shape whose upper and lower ends are both open. A center axis of the outer electrode 110 extends in the vertical direction, and corresponds to a center axis of the whole casing (i.e. the center axis of the first casing 100 and the center axis of the second casing 200). An outer surface of the outer electrode 110 is entirely joined to an inner surface of the whole casing. Further, an upper surface of the outer electrode 110 is joined to the first casing 100, and a lower surface of the outer electrode 110 is joined to the second casing 200.

The inner electrode 120 has a columnar shape. A center axis of the inner electrode 120 extends in the vertical direction, and corresponds to the center axis of the whole casing (i.e. the center axis of the outer electrode 110). An upper surface of the inner electrode 120 is joined to the first casing 100, and a lower surface of the inner electrode 120 is joined to the second casing 200. A height of the inner electrode 120 (i.e. a length in the vertical direction) is equal to a height of the outer electrode 110. An upper end of the inner electrode 120 corresponds to an upper end of the outer electrode 110. A diameter of the inner electrode 120 is smaller than a diameter of the outer electrode 110. Therefore, the storage space SS, which has a cylindrical shape, is formed between an inner surface 110a of the outer electrode 110 and an outer surface 120a of the inner electrode 120. The outer surface 120a of the inner electrode 120 entirely faces the inner surface 110a of the outer electrode 110 through the storage space SS. This causes a capacitor to be formed by the pair of electrodes 110 and 120.

The storage space SS is a space defined by the inner surface 110a of the outer electrode 110, the outer surface 120a of the inner electrode 120, the lower surface of the first casing 100, and the upper surface of the second casing 200. The storage space SS communicates with the inflow path IF and with the outflow path OP. Therefore, the storage space SS is a space configured to store fuel flowing from the inflow path IP to the outflow path OP. A center axis of the storage space SS extends in the vertical direction, and corresponds to the center axis of the whole casing (i.e. the center axis of each of the electrodes 110 and 120). A height of the storage space SS is equal to a height of the outer electrode 110 (i.e. a height of the inner electrode 120). An upper end of the storage space SS corresponds to the upper end of the outer electrode 110 (i.e. the upper end of the inner electrode 120).

The outer electrode 110 and the inner electrode 120 are connected to the control device 80. The control device 80 includes a CPU, a memory, and the like. The control device 80 uses the liquid property sensor 60 to detect a concentration of ethanol in the fuel.

As shown in FIG. 3, the inflow path IP guides the fuel from one side (i.e. the left side of FIG. 3) toward the other side (i.e. the right side of FIG. 3) in a horizontal direction. An end of the inflow path IP on the other side includes an inclined surface 304 inclined. toward the storage space SS (i.e. upward). The inclined surface 304 allows the fuel inside the inflow path IP to smoothly flow toward the storage space SS.

The outflow path OP guides the fuel from the other side (i.e. the right side of FIG. 3) to the one side (i.e. the left side of FIG. 3) in the horizontal direction. An end of the outflow path OP on the other side includes an inclined surface 314 inclined toward the storage space SS (i.e. downward). The inclined surface 314 allows the fuel inside the storage space SS to smoothly flow toward the outflow path OP.

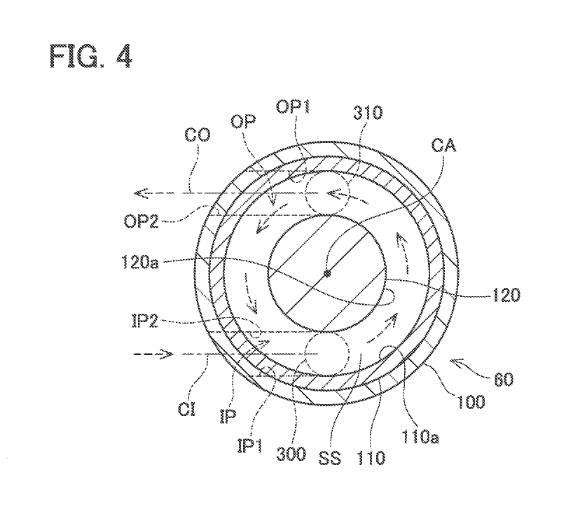

As shown in FIG. 4, in a plane vertical to an axis direction of the storage space SS, the inflow path IP are configured to allow the fuel to flow from outside the outer electrode 110 to inside the storage space SS. A first boundary IP1 of the inflow path IP (i.e. a lower boundary shown in FIG. 4) corresponds to a tangent to the outer electrode 110. A second boundary IP2 of the inflow path IP (i.e. an upper boundary shown in FIG. 4) corresponds to a tangent to the inner electrode 120. For this reason, a center line CI extending along (i.e., parallel) to the boundary IP1 and IP2 at equal distance from the boundary IP1 and IP2 does not pass through a center axis CA of the storage space SS. More specifically; the center line CI of the inflow path IP intersects the inner surface 110a of the outer electrode 110 without intersecting the outer surface 120a of the inner electrode 120.

In the present embodiment, the fuel inside the storage space SS forms a flow revolving along a circumferential direction of the storage space SS (i.e. a flow revolving counterclockwise in FIG. 4), as the inflow path IP has an ingenious shape as described above. Moreover, as shown in FIGS. 2 and 3, the fuel inside the storage space SS forms a flow from the lower side in the vertical direction to the upper side in the vertical direction, as the inflow path IP and the outflow path OP are provided on the lower side in the vertical direction and the upper side in the vertical direction, respectively. That is, the fuel inside the storage space SS forms a spiral flow including a flow from the lower side in the vertical direction to the upper side in the vertical direction and a flow revolving along a circumferential direction of the storage space SS.

A first boundary OP1 of the outflow path OP (i.e. an upper boundary shown in FIG. 4) corresponds to a tangent to the outer electrode 110. A second boundary 0P2 of the outflow path OP (i.e. a lower boundary shown in FIG. 4) corresponds to a tangent to the inner electrode 120. For this reason, a center line CO extending parallel to the boundary OP1 and OP2 at equal distance from the boundary OP1 and OP2 does not pass through the center axis CA of the storage space SS. More specifically, the center line CO of the outflow path OP intersects the inner surface 110a of the outer electrode 110 without intersecting outer surface 120a of the inner electrode 120.

In the present embodiment, the fuel forming a spiral flow can properly flow out of the storage space SS, as the outflow path OP has an ingenious shape e as described above.

### Operation of the Fuel Supplying Unit 1

When a driver starts the automobile, the fuel supplying unit 1 of FIG. 1 starts to operate. Once the fuel supplying unit 1 starts being driven, the fuel inside the reserve cup 20 passes through the low-pressure filter 32 and is sucked through the suction opening 34a into the pump body 34. Since the low-pressure filter 32 removes foreign bodies, the foreign bodies can be inhibited from entering the pump body 34. The fuel inside the pump body 34 is pressurized by an impeller provided in the pump body 34 and forced into the high-pressure filter 36 through the outlet 34b. The fuel inside the high-pressure filter 36 is sent to the pipe 50 after being filtered by a filter member. After that, the fuel passes through the pressure regulator 40 and the pipe 52 and is supplied to the engine through the discharge port 12.

The pressure regulator 40 releases the extra fuel inside the pipe 50 into the pipe 54. The fuel inside the pipe 54 flows into the storage space SS through the inflow path IP (see FIGS. 2 to 4) inside the liquid property sensor 60. The fuel inside the storage space SS forms a spiral flow including a flow from the lower side to the upper side and a flow revolving along a circumferential direction. As compared with the case where the fuel inside the storage space SS forms a linear flow, the present embodiment brings about the following effect. That is, the fuel inside the storage space SS flows toward the outflow path OP while being stirred by the spiral flow. This in turn makes it possible to inhibit the fuel from being retained inside the storage space SS. Further, since the fuel can be inhibited. from being retained, bubbles in the fuel can be properly discharged out of the storage space SS. Especially, in the present embodiment, the fuel inside the storage space SS does not form a flow from the upper side in the vertical direction to the lower side in the vertical direction but forms a flow from the lower side in the vertical direction to the upper side in the vertical direction, the bubbles in the fuel can be more properly discharged.

While the fuel supplying unit 1 is being driven, the control device 80 uses the liquid property sensor 60 to detect the concentration of ethanol contained in the fuel. The control device 80 repeats the detection of the concentration of ethanol until the engine of the automobile is stopped.

Specifically, the control device 80 is supplied with electric power from a battery (not illustrated), converts the electric power into a signal (i.e. an alternating current) of a predetermined frequency (e.g. 10 Hz to 3 MHz), and supplies the signal to one of the pair of electrodes 110 and 120. The signal supplied to the one of the pair of electrodes returns to the control device 80 from the other one of the pair of electrodes. As a result of this, charges are stored on the pair of electrodes 110 and 120, so that a capacitance is generated. The control device 80 calculates the capacitance between the pair of electrodes 110 and 120 by using the signal having returned to the control device 80 from the other one of the pair of electrodes. Further, the control device 80 detects the temperature of the fuel inside the storage space SS by using a thermistor (not illustrated) or the like.

Since a space between the pair of electrodes 110 and 120 is filled with fuel, the capacitance between the pair of electrodes 110 and 120 fluctuates in a correlated way with the dielectric constant of the fuel. Since gasoline and ethanol differ greatly in dielectric constant from each other, the dielectric constant of the fuel changes with the concentration of ethanol. Further, the dielectric constant of the fuel also fluctuates in a correlated way with the temperature of the fuel. The control device 80 includes a circuit for specifying the capacitance by using the signal having returned to the control device 80 from the other one of the pair of electrodes, a circuit for converting the capacitance into the dielectric constant of the fuel, and a database for calculating the concentration of ethanol in the fuel from the dielectric constant of the fuel and from the temperature of the fuel. These components enable the control device 80 to detect the concentration of ethanol in the fuel. Moreover, the control device 80 outputs the concentration of ethanol thus detected to an ECU (which is an abbreviation of “engine control unit”). The ECU adjusts, in accordance with the concentration of ethanol in the fuel, the amount of fuel that is to be supplied to the engine.

### Effects of the Present Embodiment

If fuel is retained inside the storage space SS, old fuel will be kept remaining in part of the storage space SS. In this case, there is a possibility that the concentration of ethanol in fuel that is newly injected into the fuel tank 10 may not be properly detected. For example, the concentration of ethanol in fuel is not necessarily constant for every fueling. For this reason, if the concentration of ethanol in fuel that is newly injected into the fuel tank 10 cannot be properly detected, it becomes impossible to supply an appropriate amount of fuel to the engine. Further, if fuel is retained in the storage space SS, it becomes hard to discharge bubbles in the fuel out of the storage space SS. This may make it impossible to properly detect the concentration of ethanol in the fuel.

In the present embodiment, as described above, the fuel inside the storage space SS forms a spiral flow. This configuration makes it possible to better inhibit the fuel from being retained inside the storage space SS than does the configuration in which the fuel inside the storage space SS forms a linear flow. Therefore, use of the liquid property sensor 60 makes it possible to properly detect the concentration of ethanol in the fuel.

### Correspondence Relationships

In the present embodiment, the outer electrode 110 and the inner electrode 120 are an example of the “first outer member” and an example of the “inner member”, respectively. The whole of the outer electrode 110 and the whole of the inner electrode 120 are an example of the “first electrode portion” and an example of the “second electrode portion”, respectively. The lower side in the vertical direction and the upper side in the vertical direction are an example of the “first side” and an example of the “second side”, respectively.

### Modifications of the First Embodiment

(1) in a plane vertical to the axis direction of the storage space SS (i.e. in a cross-section. shown in FIG. 4), the center line CI of the inflow path IP does not need to be parallel to the center line CO of the outflow path OP. Further, the center line CI of the inflow path IP may pass through the outer surface 120a of the inner electrode 120. Generally speaking, the “inflow path” needs only include a center line not passing through the center axis of the storage space.

(2) In the plane vertical to the axis direction of the storage space SS (i.e. in the cross-section shown in FIG. 4), the center line CO of the outflow path OP may pass through the outer surface 120a of the inner electrode 120. Further, the center line CO of the outflow path OP may pass through the center axis CA of the storage space SS. Generally speaking, the “outflow path” needs only be provided.

### Second Embodiment

Points of difference between the second embodiment and the first embodiment are described. As shown in FIG. 5, the second casing 220 of the liquid property sensor 60 of the present embodiment differs in configuration from its counterpart of the liquid property sensor 60 of the first embodiment. The second casing 220 includes a housing space 224 disposed on an upper stream side of the storage space SS. Further, the second casing 220 includes an inflow opening 222. The inflow opening 222 communicates with the pipe 54 (see FIG. 1) and with the housing space 224. Therefore, the inflow opening 222 allows the fuel supplied from the pipe 54 to flow into the housing space 224.

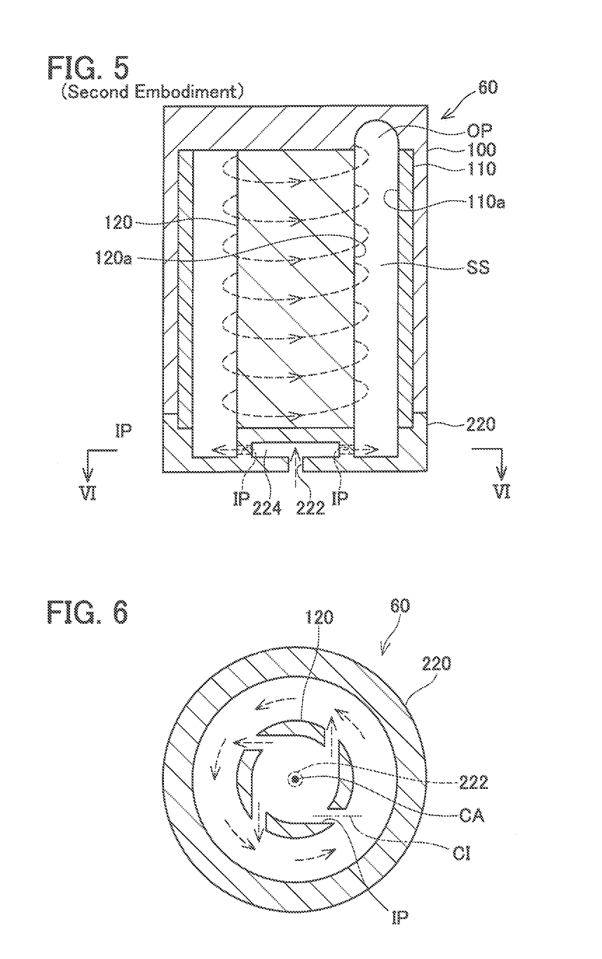

As shown in FIG. 6, the inflow opening 222 is located inside the inner electrode 120 in the plane vertical to the axis direction of the storage space SS. Further, in the present embodiment, there are provided four inflow paths IP each having a shape piercing through the second casing 220. Since such inflow paths IP each piercing through the second casing 220 are provided, the storage space SS communicates with the housing space 224 through each of the inflow paths IP. The inflow paths IP are disposed at the same position as one another in the axis direction of the storage space SS. That is, the inflow paths IP are disposed at the same height as one another.

The inflow paths IP are disposed at different positions from one another in the plane vertical to the axis direction of the storage space SS. More specifically, the inflow paths IP are disposed at regular intervals (i.e. at 90 degrees) along the circumferential direction of the storage space SS. In the plane vertical to the axis direction of the storage space SS, the center line CI of each of the inflow paths IP does not pass through the center axis CA of the storage space SS. Further, the shape of each of the inflow paths IP is set so that the fuel flows into the storage space SS in the same direction (in the present embodiment, counterclockwise) along the circumferential direction of the storage space SS. In other words, a direction in which the center line CI of each of the inflow paths IP extends is set so that when the fuel flows into the storage space SS in a specific direction (in the present embodiment, counterclockwise) along the circumferential direction of the storage space SS from one of the plurality of inflow paths IP, the fuel also flows into the storage space SS in the specific direction from all of the other inflow paths IP. This allows the fuel to properly flow in in the same direction along the circumferential direction of the storage space SS. This in turn allows the fuel inside the storage space SS to properly flow in a spiral manner.

### Effects of the Second Embodiment

In the present embodiment, too, the fuel inside the storage space SS forms a spiral flow. In particular, since there are provided a plurality of inflow paths IP, the fuel inside the storage space SS can properly flow in a spiral manner. This makes it possible to properly inhibit the fuel from being retained inside the storage space SS. In the present embodiment, the second casing 200 is an example of the “housing member”.

### Modifications of the Second Embodiment

(1) The inflow paths IF do not need to be disposed at the same position as one another in the axis direction of the storage space SS.

(2) In the plane vertical to the axis direction of the storage space SS, the inflow paths IP may be configured in either of the following manners. For example, the inflow paths IP do not need to be disposed at regular intervals along the circumferential direction of the storage space SS. Further, for example, the number of inflow paths IP does not need to be four, but needs only be one or more.

### Third Embodiment

Points of difference between the third embodiment and the first embodiment are described. As shown in FIG. 7, the first casing 130, the second casing 230, and the outer electrode 110 of the liquid property sensor 60 of the present embodiment differ in configuration from their counterparts of the liquid property sensor 60 of the first embodiment.

The second casing 230 includes a housing space 232 configured to house the fuel that is to flow into the storage space SS. The housing space 232 has a ring shape making a circle along a circumferential direction of the second casing 230. Further, the first casing 130 includes a housing space 132 configured to house the fuel that has flowed out of the storage space SS and that is to flow out of the liquid property sensor 60. The housing space 132 has a ring shape making a circle along a circumferential direction of the first casing 130.

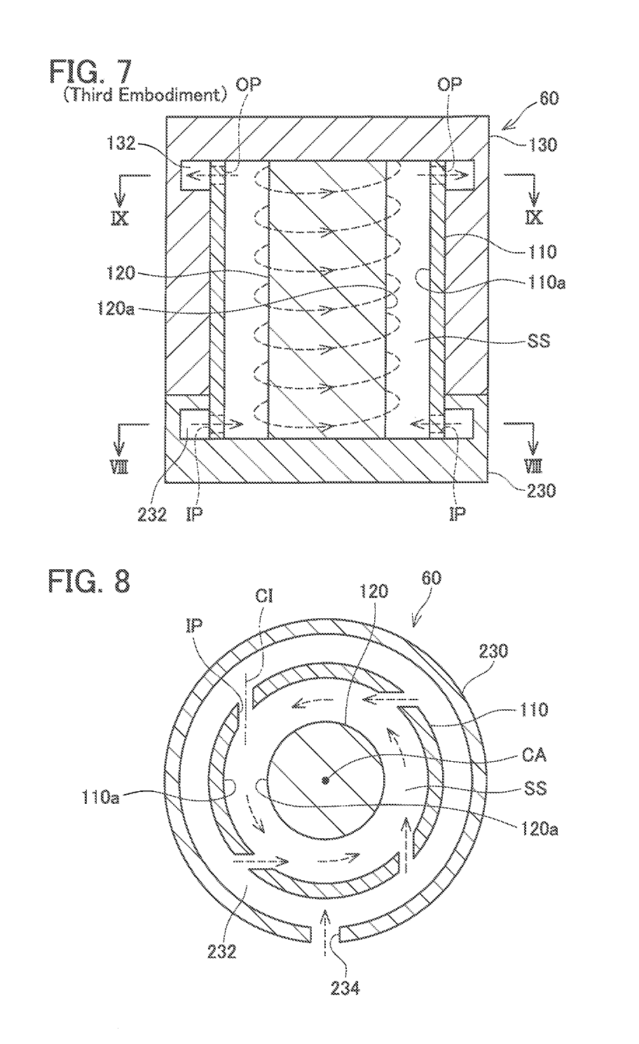

As shown in FIG. 8, the second casing 230 include an opening 234. The opening 234 communicates with the pipe 54 (see FIG. 1) and with the housing space 232. Therefore, the opening 234 allows the fuel supplied from the pipe 54 to flow into the housing space 232. In the present embodiment, there are provided four inflow paths IP each having a shape piercing through the outer electrode 110. The inflow paths IP are disposed at the same position as one another in the axis direction of the storage space SS. That is, the inflow paths IP are disposed at the same height as one another.

In the plane vertical to the axis direction of the storage space SS (i.e. in a cross-section shown in FIG. 8), the inflow paths IP are configured to allow the fuel to flow from outside the outer electrode 110 to inside the storage space SS, and are disposed at different positions from one another. More specifically, the inflow paths IP are disposed at regular intervals (i.e. at 90 degrees) along the circumferential direction of the storage space SS. The center line CI of each of the inflow paths IP does not pass through the center axis CA of the storage space SS. More specifically, the center line CI of each of the inflow paths IP intersects the inner surface 110a of the outer electrode 110 without intersecting the the outer surface 120a of the inner electrode 120. Further, the shape of each of the inflow paths IP is set so that the fuel flows into the storage space SS in the same direction (in the present embodiment, counterclockwise) along the circumferential direction of the storage space SS. This allows the fuel inside the storage space SS to properly flow in a spiral manner.

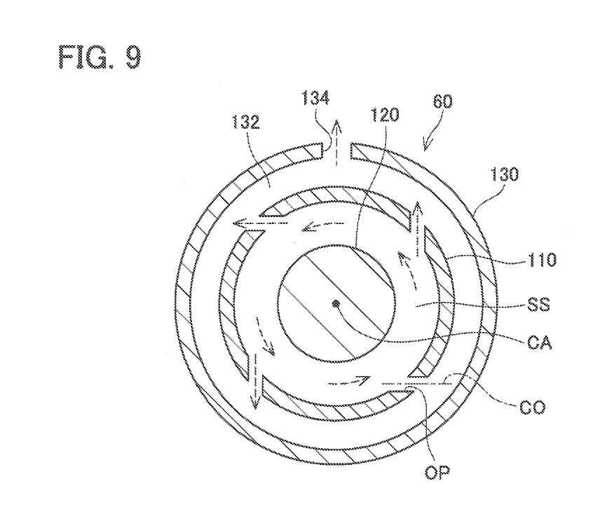

As shown in FIG. 9, the first casing 130 includes an opening 134. The opening 134 communicates with the housing space 132 and with the outside of the liquid property sensor 60 (i.e. the internal space in the fuel tank 10 (see FIG. 1)). Therefore, the opening 134 allows the fuel to flow out of the liquid property sensor 60 from the housing space 132. In the present embodiment, there are provided four outflow paths OP each having a shape piercing through the outer electrode 110. The outflow paths OP are disposed at the same position as one another in the axis direction of the storage space SS. That is the outflow paths OP are disposed at the same height as one another.

Further, the outflow paths OP are disposed at different positions from one another in the plane vertical to the axis direction of the storage space SS (i.e. in a cross-section shown in FIG. 9). More specifically, the outflow paths OP are disposed at regular intervals (i.e. at 90 degrees) along the circumferential direction of the storage space SS. The center line CO of each of the outflow paths OP does not pass through the center axis CA of the storage space SS. Further, a direction in which the center line CI of each of the outflow paths OP extends is set so that the fuel flows out of the storage space SS in the same direction as a direction (in the present embodiment, counterclockwise) in which the fuel revolves inside the storage space SS. This allows the fuel to properly flow out.

### Effects of the Third Embodiment

In the present embodiment, too, the fuel inside the storage space SS forms a spiral flow. In particular, since there are provided a plurality of inflow paths IP, the fuel inside the storage space SS can properly flow in a spiral manner. Further, since there are provided a plurality of outflow paths OP, the fuel forming a spiral flow can properly flow out of the storage space SS. This makes it possible to properly inhibit the fuel from being retained inside the storage space SS.

### Modifications of the Third Embodiment

(1) The inflow paths IP do not need to be disposed at the same position as one another in the axis direction of the storage space SS. Further, the outflow paths OP do not need to be disposed at the same position as one another in the axis direction of the storage space SS.

(2) In the plane vertical to the axis direction of the storage space SS, the inflow paths IP may be configured in the same manner as those of the modification (2) of the second embodiment. Further, in the plane vertical to the axis direction of the storage space SS, the outflow paths OP may be configured in either of the following manners. For example, the outflow paths OP do not need to be disposed at regular intervals along the circumferential direction of the storage space SS. Further, for example, the number of outflow paths OP does not need to be four, but needs only be one or more.

### Fourth Embodiment

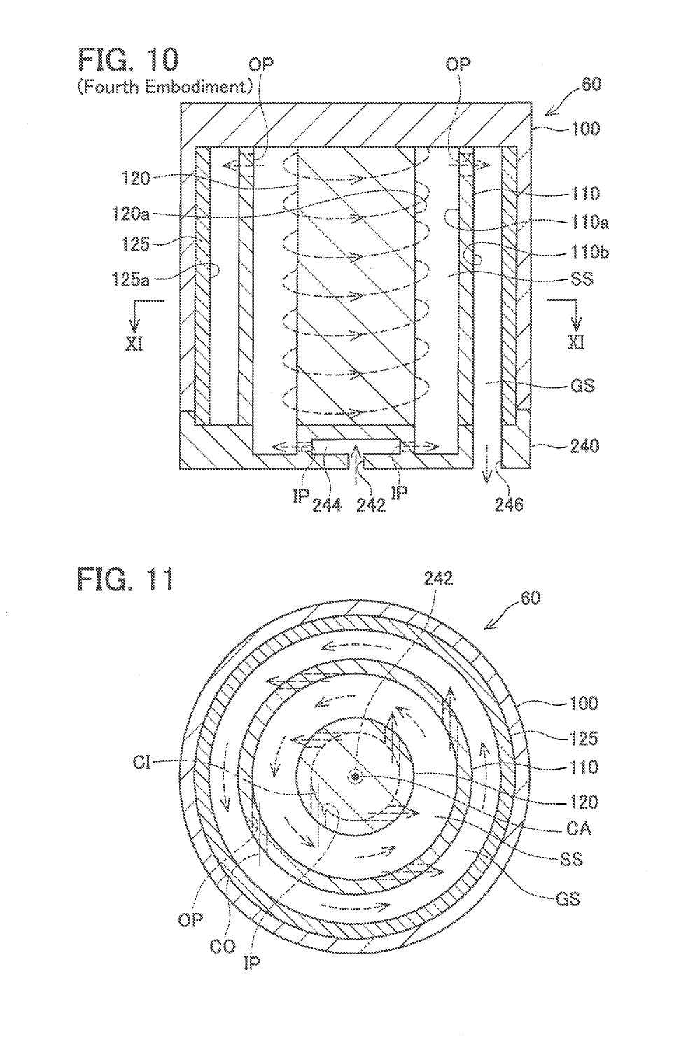

Points of difference between the fourth embodiment and the first embodiment are described. As shown in FIG. 10, the second casing 240 of the liquid property sensor 60 of the present embodiment differs in configuration from its counterpart of the liquid property sensor 60 of the first embodiment. Further, the liquid property sensor 60 further includes a guard member 125.

The second casing 240, as with its counterpart of the second embodiment (see FIG. 5), includes an inflow opening 242 and a housing space 244. Further, there are provided four inflow paths IP each having a shape piercing through the second casing 240. The inflow paths IP are configured in the same manner as those of the second embodiment. The second casing 240 further includes a discharge opening 246.

The guard member 125 is formed by a conducting material. The guard member 125 is grounded via a wire (not illustrated). The guard member 125 has a cylindrical shape whose upper and lower ends are both open. A center axis of the guard member 125 extends in the vertical direction, and corresponds to the center axis CA (see FIG. 11) of the storage space SS. An outer surface of the guard member 125 is entirely joined to an inner surface of the whole casings 100 and 240. Further, an upper surface of the guard member 125 is joined to the first casing 100, and a lower surface of the guard member 125 is joined to the second casing 240.

A height of the guard member 125 (i.e. a length in the vertical direction) is equal to the height of the outer electrode 110 (Le. the height of the inner electrode 120). An upper end of the guard member 125 corresponds to the upper end of the outer electrode 110 (i.e. the upper end of the inner electrode 120). A diameter of the guard member 125 is larger than the diameter of the outer electrode 110. Therefore, a guide space GS having a cylindrical shape is formed between an inner surface 125a of the guard member 125 and an outer surface 110b of the outer electrode 110. The outer surface 110b of the outer electrode 110 entirely faces the inner surface 125a of the guard member 125 through the guide space GS.

The guide space CS is a space defined by the inner surface 125a of the guard member 125, the outer surface 110b of the outer electrode 110, and the lower surface of the first casing 100. The guide space GS communicates with the outflow path OP and with the discharge opening 246. The guide space GS guides, to the discharge opening 246, the fuel having flowed out of the outflow path OP. That is, the guide space GS guides the fuel from the upper side to the lower side. A center axis of the guide space GS extends in the vertical direction, and corresponds to the center axis CA of the storage space SS. A height of the guide space GS is equal to the height of the guard member 125 (i.e. the height of the outer electrode 110). An upper end of the guide space GS corresponds to the upper end of the guard member 125 (i.e. the upper end of the outer electrode 110).

The discharge opening 246 allows the guide space GS and the outside of the liquid property sensor 60 (i.e. the internal space in the fuel tank 10 (see FIG. 1)) to communicate with each other. Therefore, the discharge opening 246 allows the fuel guided by the guide space GS to be discharged out of the liquid property sensor 60.

As shown in FIG. 11, there are provided four outflow paths OP each having a shape piercing through the outer electrode 110. Since such outflow paths OP each having a shape piercing through the outer electrode 110 are provided, the storage space SS communicates with the guide space GS through each of the outflow paths OP. The outflow paths OP are configured in the same manner as those of the third embodiment (see FIG. 8). The fuel inside the guide space GS forms a spiral flow including a flow from the upper side to the lower side and a flow revolving along a circumferential direction of the guide space GS (i.e. revolving counterclockwise in FIG. 11).

### Effects of the Fourth Embodiment

The present embodiment, too, makes it possible to properly inhibit the fuel from being retained inside the storage space SS. Further, in the present embodiment, the guard member 125, which is formed by a conducting member, is provided, and the guard member 125 is grounded. This makes it possible to inhibit the capacitance between the pair of electrodes 110 and 120 from fluctuating under the influence of a disturbance from outside the liquid property sensor 60. This in turn makes it possible to properly detect the capacitance between the pair of electrodes 110 and 120. In the present embodiment, the guard member 125 is an example of the “second outer member”.

### Modifications of the Fourth Embodiment

(1) The inflow paths IP and the outflow paths OP may be configured in the same manner as those of the modifications (1) and (2) of the third embodiment.

(2) The guard member 125 does not need to have a cylindrical shape, but may have a ring shape that has a polygonal cross-section. The height of the guard member 125 does not need to be equal to the height of the outer electrode 110 (i.e. the height of the inner electrode 120). Further, the guard member 125 does not need to be formed by a conducting material. Generally speaking, the “second outer member” needs only be disposed outside the first outer member and have a tubular shape.

### Fifth Embodiment

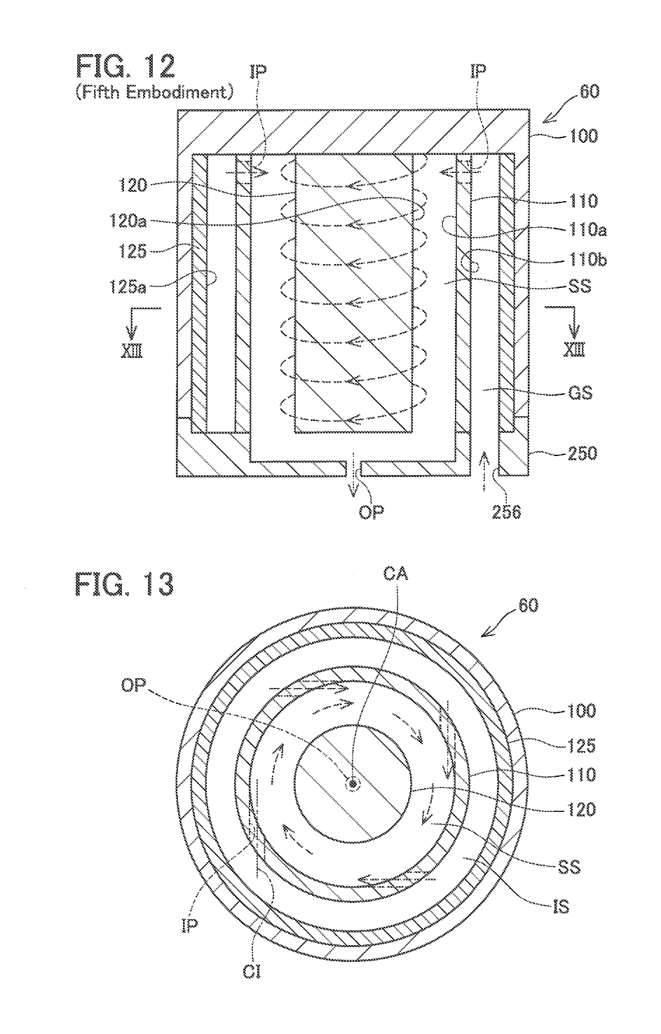

Points of difference between the fifth embodiment and the fourth embodiment are described. As shown in FIG. 12, the second casing 250 of the liquid property sensor 60 of the present embodiment differs in configuration from its counterpart of the liquid property sensor 60 of the fourth embodiment.

The second casing 250 includes an inflow opening 256 and an outflow path OP. The inflow opening 256 allows the pipe 54 (see FIG. 1) and the guide space GS to communicate with each other. Therefore, the inflow opening 256 allows the fuel supplied from the pipe 54 to flow into the guide space GS. Further, in the present embodiment, there is provided only one outflow path OP.

The guide space GS communicates with the inflow opening 256 and with an inflow path IP. Therefore, the guide space GS guides, to the inflow path IP, the fuel having flowed in through the inflow opening 256. That is, the guide space GS guides the fuel from the lower side to the upper side.

As shown in FIG. 13, there are provided four inflow paths IP each having a shape piercing through the outer electrode 110. Since such inflow paths IP each having a shape piercing through the outer electrode 110 are provided, the storage space SS communicates with the guide space GS through each of the inflow paths IP. The inflow paths IP are configured in the same manner as those of the fourth embodiment. (see FIG. 11), except that they are disposed above the outflow path OP. In the present embodiment, the fuel inside the storage space SS forms a flow revolving in the opposite direction (i.e. clockwise) to the fourth embodiment.

### Effects of the Fifth Embodiment

The present embodiment, too, makes it possible to properly inhibit the fuel from being retained inside the storage space SS. Since the guard member 125 is provided, it is possible to inhibit the capacitance between the pair of electrodes 110 and 120 from fluctuating under the influence of a disturbance from outside the liquid property sensor 60. In the present embodiment, the guard member 125 is an example of the “second outer member”. Further, the upper side in the vertical direction and the lower side in the vertical direction are an example of the “first side” and an example of the “second side”, respectively.

### Modification of the Fifth Embodiment

The inflow paths IP may be configured in the same manner as those of the modifications (1) and (2) of the third embodiment. The guard member 125 may be configured in the same manner as that of the modification (2) of the fourth embodiment.

(Modification 1) The outer electrode 110 does not need to have a cylindrical shape, but may have a ring shape that has a polygonal cross-section. Generally speaking, the “first outer member” needs only be a member having a tubular shape and configured to define a storage space for liquid. Further, the “first outer member” does not need to be formed entirely by a conducting material, but may be formed only partially by a conducting material.

(Modification 2) The inner electrode 120 does not need to have a columnar shape, but may have a ring shape that has a polygonal cross-section. Generally speaking, the “inner member” needs only be a member disposed inside the first outer member and configured to define a storage space for liquid. Further, the “inner member” does not need to be formed entirely by a conducting material, but may he formed only partially by a conducting material.

(Modification 3) The “first outer member” does not need to be an electrode formed by a conducting material, but may be formed by a non-conducting material. Similarly, the “inner member” does not need to be an electrode formed by a conducting material, but may be formed by a non-conducting material.

(Modification 4) The liquid property sensor 60 may be disposed inside the fuel tank 10 so that the axis direction of the storage space SS extends in a direction different from the vertical direction (i.e. in a horizontal direction). That is, the “first side” and the “second side” may be a first side in the direction different from the vertical direction and a second side in the direction different from the vertical direction, respectively.

(Modification 5) In each of the embodiments described above, the sensor device 2 uses the liquid property sensor 60 to detect a concentration of ethanol in fuel. However, the sensor device 2 may detect a degree of deterioration of fuel (e.g. a degree of oxidization of fuel), or may detect a property of cooling water, which is liquid other than fuel (e.g. a degree of deterioration or type of cooling water). Generally speaking, the “sensor” needs only be configured to detect a property of liquid.

(Modification 6) In each of the embodiments described above, the pipe 54 is connected to the pressure regulator 40. Alternatively, the pipe 54 may branch from the pipe 50 or may be connected to a vapor jet of the pump body 34.

(Modification 7) In each of the embodiments described above, the control device 80 detects the concentration of ethanol with the capacitance between the pair of electrodes 110 and 120 (i.e. the dielectric constant of fuel). Alternatively, the control device 80 may detect the concentration of ethanol with the dielectric constant between the pair of electrodes 110 and 120.

What is claimed is:

1. A liquid sensor configured to detect a property of liquid, the liquid sensor comprising:
a first outer member having a tubular shape;
an inner member disposed inside the first outer member;
a storage space configured to store the liquid and having a tubular shape defined by an inner surface of the first outer member and an outer surface of the inner member;
an inflow path through which the liquid flows in from outside the storage space to inside the storage space and disposed on a first side in an axis direction of the storage space; and
an outflow path through which the liquid flows out from inside the storage space to outside the storage space and disposed on a second side in the axis direction of the storage space; and
the inflow path comprises a center line not passing through a center axis of the storage space in a plane vertical to the axis direction of the storage space such that the liquid inside the storage space forms a spiral flow including a flow from the first side in the axis direction of the storage space to the second side in the axis direction of the storage space and a flow revolving along a circumferential direction of the storage space.

2. The liquid sensor as in claim 1, wherein
in the plane vertical to the axis direction of the storage space, the inflow path allows the liquid to flow in from outside the first outer member to inside the storage space, and
in the plane vertical to the axis direction of the storage space, the center line of the inflow path intersects the inner surface of the first outer member without intersecting the outer surface of the inner member,

3. The liquid sensor as in claim 1, wherein
in the plane vertical to the axis direction of the storage space, the outflow path comprises a center line not passing through the center axis of the storage space.

4. The liquid sensor as in claim 1, wherein
the first outer member comprises a first electrode portion functioning as an electrode,
the inner member comprises a second electrode portion functioning as an electrode, and
the second electrode portion opposes the first electrode portion.

5. The liquid sensor as in claim 1, further comprising:
a housing member comprising:
a housing space disposed on an upper stream side of the storage space and configured to house the liquid; and
an opening through which the liquid flows into the housing space, wherein
the opening is located inside the inner member in the plane vertical to the axis direction of the storage space,
the inflow path has a shape piercing through the housing member, and
the storage space communicates with the housing space through the inflow path.

6. The liquid sensor as in claim 1, wherein
the inflow path has a shape piercing through the outer member.

7. The liquid sensor as in claim 1, wherein
the outflow path has a shape piercing through the first outer member.

8. The liquid sensor as in claim 1, further comprising:
a second outer member disposed outside the first outer member and having a tubular shape; and
an introducing space defined by an outer surface of the first outer member and an inner surface of the second outer member and having a tubular shape, wherein
the storage space communicates with the introducing space through the inflow path and the outflow path.

9. The liquid sensor as in claim 1, further comprising:
another one or more inflow paths through which the liquid flows in from outside the storage space to inside the storage space and disposed on the first side in the axis direction of the storage space, wherein
all of the inflow paths are disposed at different positions from one another in the plane vertical to the axis direction of the storage space.

10. The liquid sensor as in claim 1, further comprising:
another one or more outflow paths through which the liquid flows out from inside the storage space to outside the storage space and disposed on the second side in the axis direction of the storage space, wherein
all of the outflow paths are disposed at different positions from one another in the plane vertical to the axis direction of the storage space.
